(19) United States
(12) Patent Application Publication
(10) Pub. No.: US 2022/0125801 A1
(43) Pub. Date: Apr. 28, 2022

(54) OPHTHALMIC COMPOSITION OF BIMATOPROST

(71) Applicant: MANKIND PHARMA LTD., New Delhi (IN)

(72) Inventors: Sundeep CHAURASIA, Manesar, Gurugram (IN); Satish Chandra UPADHYAY, Manesar, Gurugram (IN); Anil KUMAR, Manesar, Gurugram (IN)

(21) Appl. No.: 17/435,095

(22) PCT Filed: Feb. 27, 2020
(86) PCT No.: PCT/IB2020/051664

§ 371 Date: Aug. 31, 2021

(30) Foreign Application Priority Data
Mar. 6, 2019 (IN) 201911008646

(51) Int. Cl. A61K 31/5575; A61K 9/00; A61P 27/06; A61K 47/34

(52) U.S. Cl. CPC A61K 31/5575; A61K 47/34; A61P 27/06; A61K 9/0048

(57) ABSTRACT

The present invention relates to an ophthalmic composition comprising bimatoprost or pharmaceutically acceptable salts thereof and one or more pharmaceutically acceptable excipients. It further relates to a method of preparing such compositions and their use for the reduction of elevated intraocular pressure in patients with open angle glaucoma or ocular hypertension.

### FIELD OF THE INVENTION

The present invention relates to an ophthalmic composition comprising bimatoprost or pharmaceutically acceptable salts thereof and one or more pharmaceutically acceptable excipients. It further relates to a method of preparing such compositions and their use for the reduction of elevated intraocular pressure in patients with open angle glaucoma or ocular hypertension.

### BACKGROUND OF THE INVENTION

Bimatoprost is chemically known as (Z)-7-[(1R,2R,3R,5S)-3,5-Dihydroxy-2-[(1E,3S)-3-hydroxy-5-phenyl1-pentenyl]cyclopentyl]-5-N-ethylheptenamide. Bimatoprost is currently marketed in two strengths 0.03% w/v and 0.01% w/v concentration, under the brand name Latisse® and Lumigan® respectively. Both the formulations contain benzalkonium chloride as preservative and sodium phosphate as buffer. Latisse® (bimatoprost ophthalmic solution) 0.03% is indicated to treat hypotrichosis of the eyelashes by increasing their growth including length, thickness and darkness. Lumigan® (bimatoprost ophthalmic solution) 0.01% is indicated for the reduction of elevated intraocular pressure in patients with open angle glaucoma or ocular hypertension. Currently marketed 0.03% formulation contains 0.005% w/v of benzalkonium chloride, while 0.01% formulation contains 0.02% w/v of benzalkonium chloride.

U.S. Pat. No. 8,933,120 discloses an ophthalmic composition comprising about 0.01%-0.015% w/v bimatoprost and about 0.02% w/v benzalkonium chloride.

U.S. Pat. Nos. 7,851,504; 8,338,479; 8,309,605 and 8,586,630 disclose composition comprising about 0.01% bimatoprost, about 200 ppm benzalkonium chloride, a phosphate buffer, citric acid monohydrate, sodium chloride, ethylenediaminetetraacetic acid, water and having a pH of about 7.3 and its use for treating glaucoma or intraocular hypertension.

U.S. Pat. Nos. 8,278,353; 8,299,118; 8,933,127; 8,524,777; 8,772,338 and 9,155,716 disclose method of lowering intraocular pressure in a person with glaucoma or ocular hypertension, the method comprising administering once daily a first composition comprising about 0.01% w/v bimatoprost and about 0.02% w/v benzalkonium chloride, wherein the method results in improved ocular surface tolerability and less ocular adverse events such as hyperemia as compared to the once daily administration of a second composition comprising 0.03% w/v bimatoprost and 0.005% w/v benzalkonium chloride.

U.S. Pat. No. 9,241,918 discloses an ophthalmic composition for the treatment of glaucoma or elevated intraocular pressure comprising about 0.01% w/v bimatoprost, about 200 ppm benzalkonium chloride, about 0.268% w/v dibasic sodium phosphate heptahydrate, about 0.014% w/v citric acid monohydrate, about 0.81% w/v sodium chloride, and water.

U.S. Pat. Nos. 8,343,949 and 8,691,802 disclose a composition comprising 0.03% bimatoprost, 0.39% sodium chloride, 0.6% boric acid, 0.045% sodium borate, 0.014% citric acid monohydrate, 0.5% carboxymethylcellulose, and 0.005% stabilized chlorine dioxide, wherein the pH is adjusted to 7.3 by the addition of hydrochloric acid or sodium hydroxide. It also discloses an ophthalmic composition consisting of about 0.03% bimatoprost, about 0.01% Purite™, about 0.268 sodium phosphate dibasic, about 0.014% citric acid, about 0.83% sodium chloride, HCl/NaOH and purified water.

U.S. Pat. Nos. 8,268,299 and 8,323,630 disclose multi-dose, self-preserved ophthalmic composition, comprising: zinc ions, borate and polyol, the borate being present in the composition at a concentration of 0.1 to 2.0% w/v; wherein the composition has a concentration of anionic species less than 15 mM. It is disclosed that self-preserved compositions of the present invention preferably contain one or more borates in an amount of from about 0.1 to about 2.0% w/v, more preferably 0.3 to 1.5% w/v, and most preferably 0.5 to 1.2% w/v.

U.S. Patent Publication No. 2014/294750 discloses multi-dose ophthalmic composition comprising: a first polyol, the first polyol being selected from mannitol, sorbitol or a combination thereof; a second polyol, the second polyol being selected from propylene glycol, glycerine or a combination thereof; an effective amount of borate, the effective amount being less than about 0.5 w/v % of the overall composition; an antimicrobial preservative; and water. It also discloses that typically, for the present invention, the borate is at least about 0.05 w/v %, more typically at least about 0.1 w/v % and still more typically at least about 0.25 w/v % of the ophthalmic composition. Furthermore, the borate can advantageously be less than about 0.75 w/v %, more typically less than about 0.5 w/v % and still more typically less than about 0.4 w/v %, and even possibly less than about 0.35 w/v % of the ophthalmic composition.

Indian Patent Publication No. 2017/21012499 discloses ophthalmic solution comprising bimatoprost, a biguanide compound and an acylated amino acid.

Borate buffered solutions are conventionally considered bacteriostatic, whereas phosphates have been considered to be nutrients for microbes. It is known that Boric acid or other Borate buffers possess the additional feature of microbistatic inhibition in the presence of low concentrations of microbes, therefore, borate buffers are commonly used in eye drops.

It has also been reported that the use of Phosphate buffer leads to development of corneal calcification of the eye.

The object of the present invention is to provide an alternate pharmaceutical composition of bimatoprost which is free of phosphate buffer.

### SUMMARY OF THE INVENTION

The present invention relates to an ophthalmic composition comprising bimatoprost or pharmaceutically acceptable salts thereof and one or more pharmaceutically acceptable excipients.

The present invention further relates to an ophthalmic composition comprising bimatoprost or pharmaceutically acceptable salts thereof and one or more pharmaceutically acceptable excipients, where in the composition comprises less than 0.02% w/v benzalkonium chloride.

The present invention further relates to an ophthalmic composition comprising bimatoprost or pharmaceutically acceptable salts thereof and one or more pharmaceutically acceptable excipients, wherein the composition is free of phosphate buffer.

The present invention further relates to an ophthalmic composition comprising bimatoprost or pharmaceutically acceptable salts thereof and one or more pharmaceutically acceptable excipients, wherein the composition is free of preservative.

The present invention further relates to an ophthalmic composition comprising bimatoprost or pharmaceutically acceptable salts thereof and one or more pharmaceutically acceptable excipients, wherein the composition is free of benzalkonium chloride.

The present invention further relates to an ophthalmic composition comprising bimatoprost or pharmaceutically acceptable salts thereof and one or more pharmaceutically acceptable excipients, wherein the composition is free of sodium chloride.

The present invention further relates to an ophthalmic composition comprising bimatoprost or pharmaceutically acceptable salts thereof and one or more pharmaceutically acceptable excipients, wherein the one or more pharmaceutically acceptable excipients are selected from the group comprising buffers, tonicity adjusting agents, preservatives, chelating agents, antioxidants, surfactants, co-solvents, viscosity modifying agent, vehicles, pH adjusting agents and/or combinations thereof.

The present invention further relates to a method for the reduction of elevated intraocular pressure in patients with open angle glaucoma or ocular hypertension, wherein the method comprises administering a pharmaceutical composition comprising bimatoprost or pharmaceutically acceptable salts thereof and one or more pharmaceutically acceptable excipients.

The present invention further relates to a process for preparing an ophthalmic composition comprising bimatoprost or pharmaceutically acceptable salts thereof and one or more pharmaceutically acceptable excipients.

### DETAILED DESCRIPTION OF THE INVENTION

The present invention relates to an ophthalmic composition comprising bimatoprost or pharmaceutically acceptable salts thereof and one or more pharmaceutically acceptable excipients.

Another aspect of the present invention, there is provided an ophthalmic composition comprising bimatoprost and one or more pharmaceutically acceptable excipients, wherein the composition comprises from 0.005% w/v to 0.03% w/v bimatoprost or pharmaceutically acceptable salts thereof.

Another aspect of the present invention, there is provided an ophthalmic composition comprising bimatoprost or pharmaceutically acceptable salts thereof and one or more pharmaceutically acceptable excipients, wherein the bimatoprost is present in any known polymorphic form i.e. amorphous or crystalline form.

Another aspect of the present invention, there is provided an ophthalmic composition comprising bimatoprost and one or more pharmaceutically acceptable excipients, wherein the composition comprises 0.01% w/v bimatoprost or pharmaceutically acceptable salts thereof.

Another aspect of the present invention, there is provided an ophthalmic composition comprising bimatoprost and one or more pharmaceutically acceptable excipients, wherein the composition has a pH in the range of about 6.0 to 8.0, preferably in the range of 6.8 to 7.8.

Another aspect of the present invention, there is provided an ophthalmic composition comprising bimatoprost or pharmaceutically acceptable salts thereof and one or more pharmaceutically acceptable excipients, wherein the composition is in the form of clear solution, emulsion, suspension, dispersion, gel, or nanodispersion.

Another aspect of the present invention, there is provided an ophthalmic composition comprising bimatoprost or pharmaceutically acceptable salts thereof and one or more pharmaceutically acceptable excipients, where in the composition comprises 0.01% w/v bimatoprost or pharmaceutically acceptable salts thereof and less than 0.02% w/v benzalkonium chloride.

Another aspect of the present invention, there is provided an ophthalmic composition comprising bimatoprost or pharmaceutically acceptable salts thereof and one or more pharmaceutically acceptable excipients, where in the composition comprises 0.01% w/v bimatoprost or pharmaceutically acceptable salts thereof and less than 200 ppm benzalkonium chloride.

Another aspect of the present invention, there is provided an ophthalmic composition comprising bimatoprost or pharmaceutically acceptable salts thereof and one or more pharmaceutically acceptable excipients, wherein the composition is free of preservative.

Another aspect of the present invention, there is provided an ophthalmic composition comprising bimatoprost or pharmaceutically acceptable salts thereof and one or more pharmaceutically acceptable excipients, wherein the composition is free of benzalkonium chloride.

Another aspect of the present invention, there is provided an ophthalmic composition comprising bimatoprost or pharmaceutically acceptable salts thereof and one or more pharmaceutically acceptable excipients, wherein the composition is free of phosphate buffer.

Another aspect of the present invention, there is provided an ophthalmic composition comprising bimatoprost or pharmaceutically acceptable salts thereof and pharmaceutically acceptable buffer, wherein the buffer is boric acid/sodium borate buffer combination.

Another aspect of the present invention, there is provided an ophthalmic composition comprising bimatoprost or pharmaceutically acceptable salts thereof and one or more pharmaceutically acceptable excipients, wherein the composition is free of tonicity adjusting agent.

Another aspect of the present invention, there is provided an ophthalmic composition comprising bimatoprost or pharmaceutically acceptable salts thereof and one or more pharmaceutically acceptable excipients, wherein the composition is free of sodium chloride.

Another aspect of the present invention, there is provided an ophthalmic composition comprising bimatoprost or pharmaceutically acceptable salts thereof and pharmaceutically acceptable tonicity adjusting agent, wherein the tonicity adjusting agent is glycerine.

Another aspect of the present invention, there is provided an ophthalmic composition comprising bimatoprost or pharmaceutically acceptable salts thereof and one or more pharmaceutically acceptable excipients, wherein said composition is filled into a single-dose container.

Another aspect of the present invention, there is provided an ophthalmic composition comprising bimatoprost or pharmaceutically acceptable salts thereof and one or more pharmaceutically acceptable excipients, wherein said composition is filled into a multi-dose container.

Suitable containers include, for example, bottles, vials or ampoules.

Another aspect of the present invention, there is provided an ophthalmic composition comprising bimatoprost or pharmaceutically acceptable salts thereof and one or more pharmaceutically acceptable excipients, wherein said composition is filled in three piece bottle of suitable capacity plugged with nozzle and seal with cap.

Pharmaceutically acceptable packaging materials include but are not limited to low density polyethylene (“LDPE”), high density polyethylene (“HDPE”), polypropylene, polystyrene, polycarbonate, polyesters (such as polyethylene terephthalate and polyethylene naphthalate), nylon, polyvinyl chloride, poly(vinylidine chloride), poly(tetrafluoroethylene) and other materials known to those of ordinary skill in the art.

Typically the bottle may be made from Low Density Polyethylene (LDPE), Linear Low Density Polyethylene (LLDPE), High Density Polyethylene (HDPE), Polypropylene (PP) and the like or a combination thereof. Typically the nozzle/cap may be made of low density polyethylene (LDPE), linear low density polyethylene (LLDPE), high density polyethylene (HDPE), polypropylene (PP) and the like or a combination thereof. Alternatively the nozzle may be made of a hydrophobic material or may be coated with a hydrophobic material such as a fluoropolymer like Teflon (polytetrafluoroethylene) and the like.

Another aspect of the present invention, there is provided an ophthalmic composition comprising bimatoprost or pharmaceutically acceptable salts thereof and one or more pharmaceutically acceptable excipients, wherein said composition is filled in High Density Polyethylene (HDPE) bottle of suitable capacity plugged with nozzle and seal with cap.

The pharmaceutical compositions as per the present invention can be sterilized using any of the known methods of sterilization, such as filtration, moist heat, dry heat, gas sterilization or irradiation (gamma and electron beam). The container in which composition is filled can be sterilized using gamma irradiation or ethylene oxide or pre-acetic acid or any other conventional method of sterilization.

Another aspect of the present invention, there is provided an ophthalmic composition comprising bimatoprost or pharmaceutically acceptable salts thereof and one or more pharmaceutically acceptable excipients, wherein the composition has total impurities less than the maximum allowed limit as per ICH guidelines.

Another aspect of the present invention, there is provided an ophthalmic composition comprising bimatoprost or pharmaceutically acceptable salts thereof and one or more pharmaceutically acceptable excipients, wherein the one or more pharmaceutically acceptable excipients are selected from the group comprising buffers, tonicity adjusting agents, preservatives, chelating agents, antioxidants, surfactants, co-solvents, viscosity modifying agent, vehicles, pH adjusting agents and/or combinations thereof.

Suitable preservatives include cetrimide, polyquaternium-1, thiomersal or thimerosal, acids and their pharmaceutically acceptable salts such as sorbic acid, potassium sorbate, boric acid, borax, sodium perborate, sodium borate, salicylic acid, benzoic acid, lactic acid, acetic acid; S.O.C (stabilized oxychloro complex), S.C.P (stabilized chlorite peroxide), chlorobutanol, phenylethanol, benzalkonium chloride, benzododecinium bromide (BDD), cetrimonium chloride, methyl parahydroxybenzoate, polyquaternium ammonium chloride, poly aminopropyl, and hydrogen peroxide, parabens such as methyl paraben, propyl paraben, ethyl paraben, butyl paraben, perborates, phenol and its derivatives such as m-cresol and chlorocresol, benzyl alcohol, halogenated alcohols such as chlorobutanol and/or combinations thereof.

Suitable viscosity modifying agents include cellulose based polymers like hydroxypropyl methylcellulose, hydroxyethyl cellulose, hydroxypropyl cellulose, methylcellulose, carboxymethylcellulose, vinyl pyrrolidone polymers like polyvinyl pyrrolidones, or other agents like hydroxyethyl starch or dextran and/or combinations thereof.

Suitable buffering agents include, but are not limited to, phosphates, such as sodium phosphate monobasic, sodium phosphate dibasic, citric acid, citrates such as sodium citrate, acetic acid, acetates such as sodium acetate, lactic acid, sodium lactate, tartaric acid, sodium tartrate, borates including boric acid and its salts, such as sodium borate or potassium borate. Borate buffers also include compounds such as potassium tetraborate or potassium metaborate that produce borate acid or its salt in solutions, tartrates, succinates, amino acids such as L-arginine, tromethamine and/or combinations thereof.

Suitable osmotic/tonicity adjusting agents include propylene glycol, glycerol/glycerine, sodium chloride, potassium chloride, sodium bromide, calcium chloride, mannitol, sorbitol, dextrose, sucrose, mannose and the like and mixtures thereof. The osmotic agent is used in an amount to maintain the solution's osmolality compatible with respect to eye fluid.

Suitable pH adjusting agents include acids and bases. Suitable acids to adjust the pH of the composition include, but are not limited to, hydrochloric acid, citric acid, sulfuric acid, nitric acid and/or combinations thereof.

Suitable bases to adjust the pH of the composition include, but are not limited to, sodium hydroxide, potassium hydroxide, barium hydroxide, amino acid and/or combinations thereof.

Suitable vehicles/diluents include water for injection, purified water, Ringer's solution, normal saline solution. Other vehicles may be chosen, as is known in the art, including but not limited to: water soluble polyethers such as polyethylene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and/or combinations thereof.

Suitable antioxidants include, but are not limited to, sodium thiosulfate, sodium bisulfite, acetone sodium bisulfite, gentisic acid, gentisic acid ethanolamide, sodium formaldehyde sulfoxylate, thiourea, butylated hydroxyanisole, butylated hydroxy toluene, esters of gallic acid, ascorbic acid, salts of ascorbic acids such as ascorbyl palmitate, sodium ascorbate, retinoids and derivatives of vitamin A, acetylcysteine, thioglycerol, Vitamin E and its derivatives e.g. Vitamin E TPGS and/or combinations thereof.

Suitable surfactants include but are not limited to, hydrophilic surfactants, lipophilic surfactants, and mixtures thereof. The formulations can contain surface-active agents conventionally employed in topical formulations, such as oleic acid, lecithin, sorbitan trioleate, benzododecinium bromide, cetylpyridinium chloride, polyoxyethylene sorbitan monolaurate, polyoxyethylene sorbitan monostearate, polyoxyethylene sorbitan mono-oleate, polyoxypropylene/polyoxyethylene block copolymers, polyoxypropylene/polyoxyethylene/ethylene diamine block copolymers, ethoxylated castor oil and/or combinations thereof.

Suitable co-solvents include propylene glycol, polyethylene glycol, glycerine, glycerol and the like or mixtures thereof.

Suitable chelating agents include edetate disodium, ethylenediamine tetracetic acid, edetic acid, disodium edetate dihydrate, diethylenetriamine pentaacetic acid and/or combinations thereof.

Another aspect of the present invention relates to a method for the reduction of elevated intraocular pressure in patients with open angle glaucoma or ocular hypertension, wherein the method comprises administering a pharmaceutical composition comprising bimatoprost or pharmaceutically acceptable salts thereof and one or more pharmaceutically acceptable excipients.

Another aspect of the present invention relates to a method of reducing the side effect of benzalkonium chloride used in bimatoprost ophthalmic composition, said method comprises once-a-day topical instillation into the eye of a patient, an ophthalmic composition comprising bimatoprost or its pharmaceutically acceptable salt in an amount ranging from about 0.005% w/v to 0.02% w/v and one or more pharmaceutically acceptable excipients, wherein the composition comprises preservative other than benzalkonium chloride in an amount to maintain the preservative efficacy throughout the shelf-life of the product.

Another aspect of the present invention relates to a method of reducing the side effect of higher concentration of BKC (benzalkonium chloride) used in bimatoprost 0.01% w/v ophthalmic composition, said method comprises once-a-day topical instillation into the eye of a patient, an ophthalmic composition comprising 0.01% w/v bimatoprost or its pharmaceutically acceptable salt and one or more pharmaceutically acceptable excipients, wherein the composition comprises less than 0.02% w/v of benzalkonium chloride.

Another aspect of the present invention, there is provided an ophthalmic composition comprising 0.01% w/v bimatoprost or pharmaceutically acceptable salts thereof, 0-0.5% w/v preservative, 0.01-5% w/v buffering agent, 0-10% w/v tonicity agent, pH adjusting agent and purified water.

Another aspect of the present invention, there is provided an ophthalmic composition comprising 0.01% w/v bimatoprost or pharmaceutically acceptable salts thereof, 0.81% w/v Boric Acid, 0.495% w/v Sodium Borate, 0.99% w/v Glycerine, 0.02% w/v Benzalkonium Chloride, Sulfuric Acid, Sodium Hydroxide and Purified Water.

Another aspect of the present invention, there is provided an ophthalmic composition comprising 0.01% w/v bimatoprost or pharmaceutically acceptable salts thereof, 1.206% w/v Boric Acid, 0.737% w/v Sodium Borate, 0.02% w/v Benzalkonium Chloride, Sulfuric Acid, Sodium Hydroxide and Purified Water.

Another aspect of the present invention, there is provided an ophthalmic composition comprising 0.01% w/v bimatoprost or pharmaceutically acceptable salts thereof, 0.81% w/v Boric Acid, 0.495% w/v Sodium Borate, 0.99% w/v Glycerine, 0.02% w/v Benzalkonium Chloride, Hydrochloric Acid, Sodium Hydroxide and Purified Water.

Another aspect of the present invention, there is provided an ophthalmic composition comprising 0.01% w/v bimatoprost or pharmaceutically acceptable salts thereof, 1.206% w/v Boric Acid, 0.737% w/v Sodium Borate, 0.02% w/v Benzalkonium Chloride, Hydrochloric Acid, Sodium Hydroxide and Purified Water.

Another aspect of the present invention relates to a process for preparing an ophthalmic composition comprising bimatoprost or pharmaceutically acceptable salts thereof and one or more pharmaceutically acceptable excipients, wherein said process comprising the step of:
(a) adding buffering agent in purified water or water for injection till clear solution is obtained;
(b) optionally adding tonicity adjusting agent to the solution of step (a) and stirring to form a clear solution;
(c) optionally adding preservative to the solution of step (b); (d) adding bimatoprost to the solution of step (c) and dissolving to form the clear solution;
(e) adjusting the pH of the solution of step (d) with suitable pH adjusting agent if required;
(f) making the final volume of the solution using purified water or water for injection;
(g) optionally filtering the final solution of step (f), and
(h) filling the solution into a suitable container.

Another aspect of the present invention relates to an ophthalmic composition comprising bimatoprost or pharmaceutically acceptable salts thereof and one or more pharmaceutically acceptable excipients, wherein the composition optionally further comprises one or more additional therapeutic agents. Alternatively, a compound of this invention may be administered to a patient in need thereof in combination with the administration of one or more other therapeutic agents.

The following examples will illustrate in more detail the various aspects of the present invention.

### Example 1

| Ingredient | % (w/v) |
|---|---|
| Bimatoprost | 0.005 to 0.02 |
| Benzalkonium chloride | 0-0.02 |
| Sodium chloride | 0.1-10 |
| Sodium phosphate | 0.1-5 |
| Citric acid | 0.01-0.1 |
| Sodium hydroxide | q.s pH 6.0 to 8.0 |
| Hydrochloric acid | q.s pH 6.0 to 8.0 |
| Water for injection | q.s |

### Example 2

| Ingredient | % (w/v) |
|---|---|
| Bimatoprost | 0.005 to 0.02 |
| Sodium chloride | 0.1-10 |
| Sodium phosphate | 0.1-5 |
| Citric acid | 0.01-0.1 |
| Sodium hydroxide | q.s pH 6.0 to 8.0 |
| Hydrochloric acid | q.s pH 6.0 to 8.0 |
| Water for injection | q.s |

### Example 3

| Ingredient | % (w/v) |
|---|---|
| Bimatoprost | 0.005 to 0.02 |
| Benzalkonium chloride | 0-0.02 |
| Sodium chloride | 0.1-10 |
| Sodium acetate | 0.01-5 |
| Sodium hydroxide | q.s pH 6.0 to 8.0 |
| Hydrochloric acid | q.s pH 6.0 to 8.0 |
| Water for injection | q.s |

### Example 4

| Ingredient | % (w/v) |
|---|---|
| Bimatoprost | 0.005 to 0.02 |
| Benzododecinium bromide | 0-0.5 |
| Sodium chloride | 0.1-10 |
| Sodium phosphate | 0.1-5 |
| Citric acid | 0.01-0.1 |
| Sodium hydroxide | q.s pH 6.0 to 8.0 |
| Hydrochloric acid | q.s pH 6.0 to 8.0 |
| Water for injection | q.s |

### Example 5

| Ingredient | % (w/v) |
|---|---|
| Bimatoprost | 0.01 |
| Boric Acid | 0.81 |
| Sodium Borate | 0.495 |
| Glycerine | 0.99 |
| Benzalkonium Chloride | 0.02 |
| Sulfuric Acid | q.s pH 6.0 to 8.0 |
| Sodium Hydroxide | q.s pH 6.0 to 8.0 |
| Purified Water | q.s |

### Example 6

| Ingredient | % (w/v) |
|---|---|
| Bimatoprost | 0.01 |
| Boric Acid | 1.206 |
| Sodium Borate | 0.737 |
| Benzalkonium Chloride | 0.02 |
| Sulfuric Acid | q.s pH 6.0 to 8.0 |
| Sodium Hydroxide | q.s pH 6.0 to 8.0 |
| Purified Water | q.s |

### Example 7

| Ingredient | % (w/v) |
|---|---|
| Bimatoprost | 0.01 |
| Boric Acid | 0.81 |
| Sodium Borate | 0.495 |
| Glycerine | 0.99 |
| Benzalkonium Chloride | 0.02 |
| Hydrochloric Acid | q.s pH 6.0 to 8.0 |
| Sodium Hydroxide | q.s pH 6.0 to 8.0 |
| Purified Water | q.s |

### Example 8

| Ingredient | % (w/v) |
|---|---|
| Bimatoprost | 0.01 |
| Boric Acid | 1.206 |
| Sodium Borate | 0.737 |
| Benzalkonium Chloride | 0.02 |
| Hydrochloric Acid | q.s pH 6.0 to 8.0 |
| Sodium Hydroxide | q.s pH 6.0 to 8.0 |
| Purified Water | q.s |

Procedure:

The compositions as disclosed in above mentioned examples are prepared as per following process:
(a) adding buffering agent in purified water or water for injection till clear solution is obtained;
(b) optionally adding tonicity adjusting agent to the solution of step (a) and stirring to form a clear solution;
(c) optionally adding preservative to the solution of step (b);
(d) adding bimatoprost to the solution of step (c) and dissolving to form the clear solution;
(e) adjusting the pH of the solution of step (d) with suitable pH adjusting agent if required;
(f) making the final volume of the solution using purified water or water for injection;
(g) optionally filtering the final solution of step (f), and
(h) filling the solution into a suitable container.

Stability Studies:

Sterile, ophthalmic pharmaceutical composition of the present invention prepared according to examples 5, 6, 7 & 8 were tested for stability for 1 and 3 months at 25 C/40% RH and 40° C./25% RH in LDPE and HDPE containers placed in upright position. The results of the same are provided in tables 1-5.

TABLE 1

| Composition as per Example 5 in 2.5 mL | | | | Composition as per Example 5 in 5 mL | | |
|---|---|---|---|---|---|---|
| fill volume in 5 mL LDPE container | | | | fill volume in 10 mL HDPE container | | |
| | | 1 Month | | | 1 Month | |
| | | 25° C./40% | 40° C./25% | | 25° C./40% | 40° C./25% |
| Tests | Initial | RH_Upright | RH_Upright | Initial | RH_Upright | RH_Upright |
| Assay of | 99.7 | 98.7 | 99.9 | 99.7 | 98.6 | 99.2 |
| Bimatoprost | | | | | | |
| in % | | | | | | |

Related Substances: wt % By HPLC

| | | | | | | |
|---|---|---|---|---|---|---|
| 15 (R)- | ND | 0.136 | 0.053 | ND | ND | 0.025 |
| Isomer | | | | | | |
| 5,6-trans | ND | 0.062 | 0.039 | ND | ND | ND |
| Bimatoprost | | | | | | |
| 15-Keto | 0.015 | 0.109 | 0.114 | 0.015 | 0.027 | 0.048 |
| Bimatoprost | | | | | | |
| Bimatoprost | ND | ND | ND | ND | ND | ND |
| Ester | | | | | | |
| Any | 0.026 | 0.137 | 0.192 | — | — | — |
| unspecified | | | | | | |
| impurity | | | | | | |
| (RRT 0.47) | | | | | | |
| Any | ND | ND | 0.113 | — | — | — |
| unspecified | | | | | | |
| impurity | | | | | | |
| (RRT 1.09) | | | | | | |
| Any | — | — | — | 0.026 | 0.028 | 0.021 |
| unspecified | | | | | | |
| impurity | | | | | | |
| (RRT) | | | | | | |
| Single | 0.026 | 0.137 | 0.192 | 0.026 | 0.028 | 0.021 |
| Unknown | | | | | | |
| Max | | | | | | |
| Total | 0.09 | 0.49 | 0.66 | 0.09 | 0.06 | 0.09 |
| impurity | | | | | | |

TABLE 2

| Composition as per Example 6 in 2.5 mL | | | | Composition as per Example 6 in 5 mL | | |
|---|---|---|---|---|---|---|
| fill volume in 5 mL LDPE container | | | | fill volume in 10 mL HDPE container | | |
| | | 1 Month | | | 1 Month | |
| | | 25° C./40% | 40° C./25% | | 25° C./40% | 40° C./25% |
| Tests | Initial | RH_Upright | RH_Upright | Initial | RH_Upright | RH_Upright |
| Assay of | 99.0 | 98.9 | 98.7 | 99.0 | 98.5 | 98.4 |
| Bimatoprost | | | | | | |
| in % | | | | | | |

Related Substances: wt % By HPLC

| | | | | | | |
|---|---|---|---|---|---|---|
| 15 (R)- | ND | ND | ND | ND | ND | ND |
| Isomer | | | | | | |
| 5,6-trans | ND | ND | 0.043 | ND | ND | ND |
| Bimatoprost | | | | | | |
| 15-Keto | 0.034 | 0.094 | 0.12 | 0.034 | 0.075 | 0.087 |
| Bimatoprost | | | | | | |
| Bimatoprost | ND | ND | ND | ND | ND | ND |
| Ester | | | | | | |
| Any | 0.02 | 0.048 | 0.089 | — | — | — |
| unspecified | | | | | | |
| impurity | | | | | | |
| (RRT 0.44) | | | | | | |
| Any | ND | ND | ND | — | — | — |
| unspecified | | | | | | |
| impurity | | | | | | |
| (RRT 1.90) | | | | | | |
| Any | — | — | — | 0.02 | ND | ND |
| unspecified | | | | | | |
| impurity | | | | | | |
| Single | 0.02 | 0.048 | 0.089 | 0.02 | ND | ND |
| Unknown | | | | | | |
| Max | | | | | | |
| Total | 0.07 | 0.21 | 0.37 | 0.07 | 0.08 | 0.09 |
| impurity | | | | | | |

TABLE 3

| Composition as per Example 7 in 2.5 mL | | | | Composition as per Example 7 in 5 mL | | |
|---|---|---|---|---|---|---|
| fill volume in 5 mL LDPE container | | | | fill volume in 10 mL HDPE container | | |
| | | 1 Month | | | 1 Month | |
| | | 25° C./40% | 40° C./25% | | 25° C./40% | 40° C./25% |
| Tests | Initial | RH_Upright | RH_Upright | Initial | RH_Upright | RH_Upright |
| Assay of | 101.0 | 98.9 | 99.4 | 101.0 | 99.4 | 99.8 |
| Bimatoprost | | | | | | |
| in % | | | | | | |

Related Substances: wt % By HPLC

| | | | | | | |
|---|---|---|---|---|---|---|
| 15 (R)- | ND | 0.071 | 0.064 | ND | ND | ND |
| Isomer | | | | | | |
| 5,6-trans | ND | ND | ND | ND | ND | ND |
| Bimatoprost | | | | | | |
| 15-Keto | 0.016 | 0.066 | 0.071 | 0.016 | 0.04 | 0.043 |
| Bimatoprost | | | | | | |
| Bimatoprost | ND | ND | ND | ND | ND | ND |
| Ester | | | | | | |
| Any | 0.017 | ND | ND | — | — | — |
| unspecified | | | | | | |
| impurity | | | | | | |
| Unknown | ND | ND | 0.034 | — | — | — |
| RRT 0.28 | | | | | | |
| Unknown | ND | 0.033 | 0.037 | 0.017 | ND | ND |
| RRT 0.47 | | | | | | |
| Unknown | ND | ND | ND | — | — | — |
| RRT 0.51 | | | | | | |
| Unknown | ND | 0.027 | ND | — | — | — |
| RRT 0.84 | | | | | | |
| Unknown | ND | 0.035 | 0.062 | — | — | — |
| RRT 1.22 | | | | | | |
| Unknown | ND | ND | 0.027 | — | — | — |
| RRT 1.35 | | | | | | |
| Any | — | — | — | ND | 0.051 | ND |
| unspecified | | | | | | |
| impurity | | | | | | |
| (RRT 1.09) | | | | | | |
| Single | — | — | — | ND | ND | ND |
| Unknown | | | | | | |
| Max | | | | | | |
| Total | 0.05 | 0.23 | 0.29 | 0.05 | 0.09 | 0.04 |
| impurity | | | | | | |

TABLE 4
Composition as per Example 7 in 5 mL fill volume in 10 mL HDPE container

| | | 3 Month | | | | |
|---|---|---|---|---|---|---|
| | | | 25° C./40% | 25° C./40% | 40° C./25% | 40° C./25% |
| Tests | Initial | 5° C._Upright | RH_Upright | RH_Invert | RH_Upright | RH_Invert |
| Assay of | 101.0 | 98.1 | 98.3 | 98.2 | — | 98.8 |
| Bimatoprost | | | | | | |
| in % | | | | | | |
| 15 (R)- | ND | ND | ND | ND | — | ND |
| Isomer | | | | | | |
| 5,6-trans | ND | ND | ND | ND | — | 0.037 |
| Bimatoprost | | | | | | |
| 15-Keto | 0.016 | ND | ND | ND | — | 0.069 |
| Bimatoprost | | | | | | |
| Bimatoprost | ND | ND | ND | ND | — | ND |
| Ester | | | | | | |
| Any | 0.017 | — | — | — | — | — |
| unspecified | | | | | | |
| impurity | | | | | | |
| (RRT 0.47) | | | | | | |
| Any | ND | — | — | — | — | — |
| unspecified | | | | | | |
| impurity | | | | | | |
| (RRT 1.09) | | | | | | |
| Single | ND | ND | ND | ND | — | 0.028 |
| Unknown | | | | | | |
| Max | | | | | | |
| Total | 0.05 | ND | ND | ND | — | 0.14 |
| impurity | | | | | | |

TABLE 5

| Composition as per Example 8 in 2.5 mL | | | | Composition as per Example 8 in 5 mL | | |
|---|---|---|---|---|---|---|
| fill volume in 5 mL LDPE container | | | | fill volume in 10 mL HDPE container | | |
| | | 1 Month | | | 1 Month | |
| | | 25° C./40% | 40° C./25% | | 25° C./40% | 40° C./25% |
| Tests | Initial | RH_Upright | RH_Upright | Initial | RH_Upright | RH_Upright |
| Assay of | 99.5 | 96.7 | 98.5 | 99.5 | 97.7 | 99.0 |
| Bimatoprost | | | | | | |
| in % | | | | | | |

Related Substances: wt % By HPLC

| | | | | | | |
|---|---|---|---|---|---|---|
| 15 (R)- | ND | 0.144 | 0.04 | ND | ND | ND |
| Isomer | | | | | | |
| 5,6-trans | ND | ND | ND | ND | ND | ND |
| Bimatoprost | | | | | | |
| 15-Keto | 0.024 | 0.233 | 0.135 | 0.024 | 0.053 | 0.077 |
| Bimatoprost | | | | | | |
| Bimatoprost | ND | ND | ND | ND | ND | ND |
| Ester | | | | | | |
| Any | 0.023 | ND | ND | 0.023 | ND | ND |
| unspecified | | | | | | |
| impurity | | | | | | |
| Unknown | ND | 0.07 | 0.061 | — | — | — |
| RRT 1.22 | | | | | | |
| Unknown | — | — | — | ND | ND | ND |
| RRT 0.79 | | | | | | |
| Unknown | — | — | — | ND | ND | ND |
| RRT 1.52 | | | | | | |
| Total | 0.05 | 0.45 | 0.24 | 0.05 | 0.05 | 0.08 |
| impurity | | | | | | |

The obtained stability data showed that the compositions as per present invention are stable in both LDPE and HDPE containers. However, compositions are more stable in HDPE container.

1. A pharmaceutical composition comprising bimatoprost or pharmaceutically acceptable salts thereof and one or more pharmaceutically acceptable excipients, wherein the composition is free of phosphate buffer.

2. The pharmaceutical composition as claimed in claim 1, wherein the pharmaceutically acceptable excipients are selected from the group comprising buffering agent, tonicity adjusting agent, pH adjusting agent, vehicle/diluent/solvent, surfactant, antioxidant, preservative and/or combinations thereof.

3. The pharmaceutical composition as claimed in claim 2, wherein the buffering agent is boric acid/sodium borate buffer combination.

4. The pharmaceutical composition as claimed in claim 2, wherein the tonicity adjusting agent is selected from propylene glycol, glycerine, sodium chloride, potassium chloride, sodium bromide, calcium chloride, mannitol, sorbitol, dextrose, sucrose, mannose and/or combinations thereof.

5. The pharmaceutical composition as claimed in claim 4, wherein the tonicity adjusting agent is glycerine.

6. A pharmaceutical composition comprising bimatoprost or pharmaceutically acceptable salts thereof and one or more pharmaceutically acceptable excipients, wherein the composition is filled in a HDPE container.

7-10. (canceled)

11. The pharmaceutical composition as claimed in claim 1, wherein said pharmaceutical composition is useful for reduction of elevated intraocular pressure in patients with open angle glaucoma or ocular hypertension.

12. A process for preparing an ophthalmic composition comprising bimatoprost or pharmaceutically acceptable salts thereof and one or more pharmaceutically acceptable excipients, wherein said process comprises the steps of:
(a) adding buffering agent in purified water or water for injection till clear solution is obtained;
(b) optionally adding tonicity adjusting agent to the solution of step (a) and stirring to form a clear solution;
(c) optionally adding preservative to the solution of step (b);
(d) adding bimatoprost to the solution of step (c) and dissolving to form the clear solution;
(e) adjusting the pH of the solution of step (d) with suitable pH adjusting agent if required;
(f) making the final volume of the solution using purified water or water for injection;
(g) optionally filtering the final solution of step (f); and
(h) filling the solution into a suitable container.
